SEQUENCE LISTING (14 sequences)

SEQ ID NO: 1 - DNA, 15 bp, Artificial Sequence
  Description of Artificial Sequence Synthetic probe
atatacctcacgtcg

SEQ ID NO: 2 - DNA, 15 bp, Artificial Sequence
  Description of Artificial Sequence Synthetic probe
aggcacctctgtaag

SEQ ID NO: 3 - DNA, 15 bp, Artificial Sequence
  Description of Artificial Sequence Synthetic probe
atacacctctgtttc

SEQ ID NO: 4 - DNA, 15 bp, Artificial Sequence
  Description of Artificial Sequence Synthetic probe
atacacctcagatcg

SEQ ID NO: 5 - DNA, 15 bp, Artificial Sequence
  Description of Artificial Sequence Synthetic probe
atacacctcactccg

SEQ ID NO: 6 - DNA, 16 bp, Artificial Sequence
  Description of Artificial Sequence Synthetic probe
gtcttccaaagtacga

SEQ ID NO: 7 - DNA, 18 bp, Artificial Sequence
  Description of Artificial Sequence Synthetic probe
ttccaatttacgtatgtc

SEQ ID NO: 8 - DNA, 16 bp, Artificial Sequence
  Description of Artificial Sequence Synthetic probe
tccaaagtacgaatgt

SEQ ID NO: 9 - DNA, 15 bp, Artificial Sequence
  Description of Artificial Sequence Synthetic probe
gtatggttcgtaggt

SEQ ID NO: 10 - DNA, 18 bp, Artificial Sequence
  Description of Artificial Sequence Synthetic probe
cttgcaatatgcgaatat

SEQ ID NO: 11 - DNA, 16 bp, Artificial Sequence
  Description of Artificial Sequence Synthetic probe
tcgcagtaactgttgc

SEQ ID NO: 12 - DNA, 15 bp, Artificial Sequence
  Description of Artificial Sequence Synthetic probe
tcacgtgcgagtaac

SEQ ID NO: 13 - DNA, 15 bp, Artificial Sequence
  Description of Artificial Sequence Synthetic probe
taatacacctcacgt

SEQ ID NO: 14 - DNA, 15 bp, Artificial Sequence
  Description of Artificial Sequence Synthetic probe
gttaatacacctcac

(19) United States
(12) Patent Application Publication
(10) Pub. No.: US 2007/0111960 A1
(43) Pub. Date: May 17, 2007

(54) High affinity probes for analysis of human papillomavirus expression

(75) Inventors: Henrik Stender, Gentofte (DK); Mark Fiandaca, Princeton, MA (US)

Correspondence Address: EDWARDS & ANGELL, LLP, P.O. BOX 55874, BOSTON, MA, 02205, US

(73) Assignee: AdvanDx, Inc., Woburn, MA (US)

(21) Appl. No.: 11/516,409

(22) Filed: Sep. 5, 2006

(30) Foreign Application Priority Data
Mar. 4, 2005 (US) PCT/US05/06999

(51) Int. Cl. A61K 31/70; A01N 43/04

(52) U.S. Cl. 514/44

(57) ABSTRACT

This invention is directed toward methods kits and compositions pertaining to the use of high affinity probes for diagnostic applications. The invention is further directed toward the use of the high affinity probes for the detection of clinically important human papillomavirus (HPV) strains. High affinity probes enable rapid diagnostic assays and supply sufficient hybridization specificity to discriminate closely related HPV strains. Examples of high-affinity probes are provided which are specifically directed towards the detection of the E6 and E7 regions, and splice variants thereof of HPV mRNA.

### RELATED APPLICATION

This application contains subject matter that is related to that disclosed in provisional patent application Ser. No. 60/550,696 filed Mar. 5, 2004 and in International Patent Application No. PCT/US2005/06999, both entitled, “High Affinity Probes for Diagnosis of Cervical Cancer,” the disclosure of which application is incorporated herein in its entirety by this reference.

### BACKGROUND OF THE INVENTION

1. Field of the Invention

This invention is related to the field of probe based nucleic acid sequence detection, analysis and quantification. More specifically, this invention relates to novel kits, compositions and methods for high affinity probes, such as peptide nucleic acid (PNA) and locked nucleic acid (LNA), and their use in the detection and typing of HPV infection in human cells.

Papillomaviruses infect epithelial cells of the skin and mucous membranes, generally inducing benign tumors (warts). The greater than seventy variants of papillomaviruses which have been described in humans are arranged into three symtomologic classes, gential-mucosal, non-genital and epidermiodysplasia verruciformis (EV)-specific (Lowy, PNAS Mar. 29, 1994; 91(7): 243640). EV is a rare skin disease in which individuals develop chronic HPV lesions. Non-genital HPV genotypes cause common warts, plantar warts, and flat warts, but are otherwise clinically irrelevant. EV and non-genital types account for over half of the HPV variants identified. These variants are distributed worldwide in high prevalence.

The Papanicolaou (Pap) smear is a method of preparing and staining cells obtained from cervical scrapings to assess the risk of cervical cancer. Cells are examined microscopically by a cytologist for indicators of precancerous or cancerous cells such as variations in the size of nuclei, presence of large vacuoles (koilocyes), abnormal nuclear/cytoplasmic ratios, and hyperchromic nuclei. Pap test results are frequently entered into a scoring algorithm which incorporates these observations as well as the number and type of cells identified by Pap smear as atypical squamous cells of undetermined significance (ASCUS). Pap test histologic scoring indicates the progression of cervical epithelia pre-neoplastic lesions by differentiating them into three grades of increasing clinical severity. Cervical intraepithelial neoplasia grade 1 (CIN I) is the lowest grade, followed by CIN II and III which is the last stage before cervical carcinoma in situ. A CIN I score is also described cytologically as low grade squamous intraepithelial lesions (LSIL). CIN II and III are considered high grade squamous intraepithelial lesions (HSIL).

Due to the high false positive rate of Pap smear analysis the current follow up protocol for a positive result is a retest. Recent studies such as the ASCUS LSIL Triage Study and subsequent analyses (Schiffman M, Acta Cytol. September-October 2000;44(5):726-42, and Am J Obstet Gynecol. June 2003;188(6):1383-92) ultimately concluded that available HPV reflux testing was overall more effective at diagnosing low and high grade cervical dysplasias than repeat Pap analysis, and was possibly even more cost effective (Goldie et al, Obstet Gynecol. April 2004;103(4):619-31). Pap testing also has a high false negative rate, but use of secondary genetic testing has been shown to increase the negative predictive value to 99% (Salomon D, et al. J Natl Cancer Inst. 2001; 93: 293-9). In women >30 years old, tri-annual testing by a combination of cytological and genetic methods was shown to be more cost effective and more accurate that annual Pap testing for diagnosis of LSIL and HSIL. Although genetic HPV testing methods are proven effective as reflux tests, their stand alone value is limited by the high prevalence of HPV infection in patients who do not have lesions, and will not go on to develop them.

A few HPV types are strongly associated with the development of pre-malignant and malignant genital disease. In fact, ninety percent of cervical cancers and high-grade cervical lesions contain detectable levels of HPV DNA. Genital HPV variants are further delineated into low risk and high risk types. The low risk types (e.g. HPV6 and HPV11) are rarely associated with cervical malignancies. The high risk HPV types account for the majority of cervical cancers. HPV16 is the most frequently identified type (40-60% of cases) followed by HPV18 (10-20%), then by HPV31, HPV33, and HPV45. Genital HPV infection is perhaps the most prevalent sexually transmitted disease in the world. Individuals are often unaware of infection since visible symptoms can be easily overlooked. The onset of cervical cancer can be preceded by an incubation period of more than 20 years after the initial infection. Once diagnosed, one in three women with cervical cancer will die from the disease. When detected at an early stage, however, cervical cancer is one of the most successfully treatable cancers with a 5-year survival rate of >90%. Since the seriousness of infection is so dramatically different among HPV variants and the prevalence of HPV infection is so high, it is important to have tests to identify the most dangerous genotypes, and to further classify them as low risk or high risk types.

A hallmark of neoplastic progression is incorporation of the HPV episomal genome into the chromosomes of the host cell, a process known as integration. Though HPV variants are described as high, and low risk it has become increasingly clear that integration of any HPV variant into the genomic DNA of cervical epithelial tissues is clinically relevant. The site of genomic integration though, is not considered relevant, since it has been demonstrated that integration sites are not conserved among patients (Klaes, Oncogene, 21(3) 419426, 2002). It has also been demonstrated that genomic integration is a very rare event, which needs only to occur once to transform a cell (von Knebel Doeberitz, et. al., Oncogene 22 (25) 3977-84, 2003). Since it is integration of HPV that is the surest marker of neoplastic progression it is important that tests are developed which not only determine HPV type, but can also distinguish integrated form episomal HPV. Nucleotide sequence analysis shows seven open reading frames (ORFs) in the HPV genome. There are two “late” ORFs that encode capsid proteins, and others are “early” ORFs that are involved in viral replication and cellular transformation. Genes E6 and E7 are both required for cell transformation. The early genes, E1, E2, E3 and E4 are frequently disrupted during integration which has led to the hypothesis that loss of these early genes activates the oncogenic potential of E6 and E7. Physical differences between integrated and episomal mRNAs transcribed from their respective HPV genomes, indicates the origin of the particular mRNAs and has been used to discriminate between the two message sources (Klaes, ibid). These differences come in the form of alternate mRNA splice variants, which are observable by sequence determination, measurement of mRNA length, or hybridization profile.

Many methods and kits for specific identification of HPV types through analysis of amplified nucleic acids taken from patient samples are described in the art. Amplification achieved through the polymerase chain reaction is described in several patents including U.S. Pat. No. 5,750,334, EP0524807, and U.S. Pat. No. 5,580,970 incorporated herein by reference. Several other patents describe primers, probes and assays for use in specific identification, see U.S. Pat. No. 6,265,154, US2004018539, WO01/51501, and WO89/09940 all incorporated herein by reference. U.S. Pat. No. 5,705,627 (Hoffman-LaRoche) reports use of polymerase chain reaction (PCR) to amplify and detect HPV DNA using degenerate or mixed consensus primers, followed by typing using a mixture of genotype-specific DNA probes (see U.S. Pat. No. 5,182,377, U.S. Pat. No. 5,283,171, U.S. Pat. No. 5,447,839, U.S. Pat. No. 5,527,898, U.S. Pat. No. 5,639,871 for related art from Hoffman-LaRoche). Amplification of mRNA by the NASBA method (see WO03/020975, WO03/020976, WO03/057927) have also been described. Amplification though other methods such as rolling circle amplification, TMA, and others are claimed elsewhere, see US2003108866. Amplified assays, though sensitive, are prone to technical failures resulting in false positives and negatives, and they are often expensive and time consuming. US2003108866 also teaches the use of peptide nucleic acid (PNA) probes to specifically block PCR reactions in the presence of certain HPV genotypes. PNA probes have also been used for the analysis of rRNA in ISH and FISH assays (See: WO95/32305 (Dako) for detection of bacteria causing STDs), as well as in the analysis of mRNA (e.g. Kappa & Lambda Light Chain; Thisted M. et al., Cell Vision 3: 358-363(1996)) and small nuclear RNA (Just T et al., J. Vir. Methods: 73:163-174 (1998)).

U.S. Pat. No. 5,888,733 (Dako) teaches in situ detection of targets in samples or eukaryotic origin by PNA-FISH, including eukaryotic mRNA, bacterial rRNA, genomic RNA (of an RNA virus), and chromosomal DNA repeats. The LNA and PNA FISH technology has been extensively applied as a direct probe technology, i.e., without the need for signal amplification, within the area of microbiology using high copy rRNA as target (Stender, Microbiol Methods. 2002 48:1-17) or cytology using chromosomal repeat sequences (Taneja, Gen Chro Can 2001 30:57-63; Silahtaroglu, Cytogenet Genome Res. 2004;107(1-2):32-7) as target, whereas state of the art in situ hybridization for expression analysis using PNA probes targeting mRNA transcripts are done using signal amplification, such as catalyzed signal amplification (Larsen, Prenat Diagn 2003 23 52-9) or enzyme-labeled antibodies (Thisted, Cell Vis 1996 3:5 358-363).

US2003108866 and U.S. Pat. No. 5,888,733 both have prophetic examples for the detection of HPV by in situ hybridization using PNA probes. US2003108866 teaches a method using signal amplification, but did not envision the potential of direct detection. U.S. Pat. No. 5,888,733 does teach direct detection of RNA in the illustrative examples comprising all possible detection technologies and exemplifies direct detection using PNA probes targeting rRNA and viral genomic RNA (of an RNA virus), but for the analysis of mRNA signal amplification using enzyme-labeled antibodies were applied.

Hybridization assays combined with signal amplification have been used to detect HPV as described in U.S. Pat. No. 6,228,578 and as commercialized in the form of the Digene Hybrid Capture 2 Assay (HC2) which combined with a Pap screening test is marketed as the FDA approved DNAwithPap™ test (Digene Corp., Gaithersburg, Md.). This assay detects the formation of hybrids made between RNA probes and DNA targets (HPV) through the use of an antibody which is specific to DNA/RNA hybrids. Direct mRNA detection by sandwich assay followed by secondary signal generation has also been described WO94/26934 (Baxter). Neither of these assays can discriminate between episomal and incorporated targets.

Furthermore, methods of combining and correlating direct or amplified detection of DNA or RNA targets with detection of protein markers indicative of cellar transformation have been described, WO2004092734 (Digene).

It is however well-recognized that the presence of HPV alone is not indicative of an active infection as HPV may be transitional and disappear without any clinical manifestations and therefore not associated with the presence of ASCUS cells observed by PAP stain. Methods for determination of viral integration have a greater prognostic value since integration precedes neoplastic progression. Assays such as those described in U.S. Pat. No. 6355242 are designed to measure relative expression of viral oncogenes (E6/E7) compared to non-transforming genes (E1/E2) which are typically disrupted during the process of integration.

In situ hybridization (ISH) or Fluorescent-ISH (FISH) combines cell morphology with the sensitivity and specificity of molecular techniques and has been used to detect intracellular HPV using either cloned DNA probes CA2348413, US2005014133 (Ventana), and WO89/02934 (Microprobe) or DNA probe cocktails (WO/02/44401). Similarly, methods have been described where FISH (among other detection methods) is used for detection of high-grade dysplasia and carcinoma in cervical cells by correlation of amplification of chromosomal loci associated with oncogenesis (WO2005/001137 Vysis). ISH combined with signal amplification has also been used for this purpose (see WO01/94632 Bayer A G, example 3), including highly sensitive and specific detection of HPV RNA (see also Kenny et al, JHC refs from Bayer AG). DNA FISH combined with flow cytometry has also been described in US2004/260157 for automated evaluation of cervical specimens.

In situ hybridization to detect expression of HPV mRNA (not just integration of HPV DNA) has a high prognostic value while at the same time being a simpler assay procedure due to natural target amplification (via active transcription), and maintenance of relatively high cellular target concentrations (as compared to methods which disrupt cells to access targets). Also, HPV RNA has been shown to be a better indicator of neoplasmic progression than HPV DNA, as it progressively increases in concentration as a function of SIL grade (Wang-Johanning Caner 2002, 94:2199-210). In situ techniques also benefit from the availability of automated slide processing instruments in the marketplace.

Direct detection of the level of mRNA without the use of signal amplification would be advantageous compared to current methods. Probes designed to discriminate HPV mRNA from integrated or episomal HPV genomes, and which can determine HPV type would be particularly beneficial.

It would be desirable to have direct probes for analysis of expression of high risk human papillomavirus to support the diagnosis of cervical cancer. It would be further desirable to have an in situ hybridization method for analysis of the expression of high risk human papillomavirus in cervical specimens. Especially desirable would be to have an in situ hybridization method to analyze expression of high risk human papillomavirus in cytological specimens where abnormal cervical cells were detected by a Pap test in order to confirm active viral replication within the abnormal cells. Even more desirable would be direct high affinity probes to increase the accuracy, specificity of such assays while also increasing the time to result and ease of use of the assays. Thus it is an object of this invention to provide high affinity probes, methods and kits for direct detection of mRNA targets from integrated high risk HPV types.

### SUMMARY OF THE INVENTION

This invention is directed towards methods, kits and compositions pertaining to high affinity probes for analysis of high risk human papillomavirus types. The high affinity probes are directed towards mRNA for determination of active expression of viral genes. Probing nucleobase sequences of the high affinity probes are listed in Table 1. The invention provides probe designs and methods for selecting probes which simplifies the process of HPV detection since properly designed high affinity probes will only detect high risk HPV mRNA sequences ensuring that positive test results correlate to expression of viral genes.

In one embodiment, the invention provides method to selectively detect high grade cervical intraepithelial neoplasia (CIN II and CIN III) and malignant carcinoma in cervical biopsy and Pap smear specimens without detecting low grade cervical intraepithelial neoplasia (CIN I). The invention is based on the use of in situ hybridization in which high affinity probes are used to assay cervical samples. In one embodiment, the high affinity probes include peptide nucleic acids (PNA), locked nucleic acids (LNA), and other modified backbone probes. These probes are made by synthetic organic chemistry and are much shorter than conventional DNA probes, particularly cloned, full length, double-stranded DNA probes and therefore are both easier and cheaper to produce in a reproducible manner. The hybridization results are then correlated with a clinical diagnosis of high grade cervical intraepithelial neoplasia (CIN II and CIN III) and malignant carcinoma.

In other embodiments, the high affinity probes are based on those described in Table 1, but differ in length by shortening, or lengthening the probe sequence corresponding to the complementary bases in the target sequences, and correlating to respective decreases or increases in the stringency of the hybridization assay. The probe sequences range in length from 8 to 20 nucleobases. It is a further embodiment of this invention that probes may be used singly, or in combination with a second probe, or any number of probes.

Probes of the invention derived from the probes listed in Table 1 can be selected by i), determining target sequences of high risk HPV mRNAs which differ between integrated and episomal forms, and ii) designing high affinity probes which are substantially complementary to the mRNAs derived from integrated forms and differ by at least one base to mRNAs derived from the episomal forms. One of skill in the art having the benefit of this disclosure would understand how to select the derived probes based on an understanding of nucleic acid hybridization and methods of testing individual probe designs.

In one embodiment, the probes described are labeled with at least one detectable moiety selected from a group including, but not limited to a conjugate, a branched detection system, a chromophore, a fluorophore, a spin label, a radioisotope, an enzyme, a hapten, an acridinium ester and a luminescent compound. Alternatively, the probe or probes may be labeled with multiple detectable moieties. The multiple detectable moieties may be identical or different labels on a single probe, or they may be identical or different labels on multiple probes. In a preferred embodiment, multiple similarly labeled probes are used in combination. In another preferred embodiment, multiple type specific HPV probes covering a panel of high risk types are used in combination.

In another embodiment, the invention provides methods for the secondary detection of a hapten label on a probe or probes with an affinity label followed by subsequent detection of the aforementioned affinity label. The affinity label may include, for example, streptavidin, or a streptavidin conjugate where the hapten is biotin, or it may include an anti-hapten antibody, or anti-hapten antibody-enzyme conjugate. Examples of anti-hapten antibody-enzyme conjugates include anti-FITC-AP, anti-FITC-HRP, and anti-FITC-SBP, where the hapten is fluorescein isothiocyanate (FITC) or its derivatives, and AP, HRP, and SBP are alkaline phosphatase, horseradish peroxidase, and soybean peroxidase respectively. Where peroxidase labels are used, secondary signal amplification may be performed though the use of tyramide conjugate labels.

The probes of the invention may contain spacers, linkers, or other moieties which do not contribute significantly to the specific hybridization of the probes, but confer other benefits such as solubility enhancement, reduction of steric effects, promotion of cellular uptake, etc. Alternate probe labeling schemes include probes which are self reporting, most preferably, the self reporting probes are PNA Linear Beacons.

As illustrated in Example 3, the inventors have shown that fluorescent labeled PNA probes can be applied for direct analysis of mRNA transcript by fluorescence in situ hybridization without the requirement for signal amplification. Direct detection of mRNA without signal amplification offers the potential for simpler assay formats as well as direct correlation to the amount of mRNA targets whereas the use of signal amplification technologies may make chromosomal DNA and non-expressed/weakly genes detectable. Furthermore, direct detection facilitates the use of differently labeled high affinity probes for multicolor assays where differently labeled probes can be used for simultaneous analysis of expression of multiple genes and/or for comparative expression analysis. The discovery therefore facilitates the development of method for analysis of gene expression and by this means to either determine integration of a invasive gene, such as but not limited to E6/E7 HPV or regulation (up- or down) of naturally occurring genes, such but not limited Her2-neu, Bcl-2 and genes for growth factor receptors. Moreover, the method enables simultaneous detection of up-regulation/high expression and down-regulation/low expression, such as but not limited to higher expression of E6/E7 as compared to E1/E2, which provides a means to increase the test specificity or provide better prognostic information within a single reaction. The latter may be used to distinguish between episomal and integrated HPV and/or monitor a disease state.

In one embodiment, the invention provides methods comprising i), fixing sample cells using one of several fixation methods; ii) contacting the HPV mRNA with at least one high-affinity probe that is substantially complementary to a portion of a HPV mRNA; and iii) detecting hybridization between the probe and the mRNA. Detection of mRNA provides natural amplification of the targets via viral expression and thus facilitates development of simple assay formats, preferably without extensive use of either target or signal amplification technologies.

The invention provides methods for expression analysis of mRNA. The methods comprise contacting a cytological specimen with a high affinity probe of the invention under conditions suitable for in situ hybridization. In a related method, the invention provides methods for the expression analysis of a cancer marker, comprising contacting a cytological specimen with a high affinity probe according to the invention under conditions suitable for in situ hybridization. In another related method, the invention provides, methods for expression analysis of human papillomavirus comprising contacting a cytological specimen with a high affinity probe of any one of claims 1-22 under conditions suitable for in situ hybridization.

The invention also provides methods for the expression analysis of human papillomavirus by in situ hybridization, comprising:

a) contacting the sample with at least one high-affinity probe that is substantially complementary to a portion of a HPV mRNA.

b) incubating the sample with the high affinity probe; and

c) detecting the fluorescence of the sample, wherein the level of fluorescence is indicative of the presence and/or amount of mRNA within individual cell of the sample.

As used herein, detecting includes measuring, for example, measuring a fluorescence signal.

One aspect of the invention features a method whereby the expression of high risk HPV types in cervical specimens are detected and analyzed by in situ hybridization of high affinity probes to HPV mRNA. The method may be performed on specimens in which abnormal cells were determined by Pap stain. The cervical specimens may be prepared, for example, as ThinPrep™ or SurePath™ slides. Alternatively the test could also be used as a screening method which is performed prior to Pap staining, or which precludes the need for Pap staining. One advantage of this method is that it allows simultaneous detection of probe-target hybrids and analysis of cell morphology by microscopy. The presence of the HPV mRNA in the sample cells is diagnostic of HPV infection, and may be indicative of risks of cancer, such as risks associated with endocervical carcinoma, cervical cancer, dysplasia and neoplasia.

In one embodiment the invention provides method combining Pap staining and detection of HPV targets with fluorescently labeled high affinity probes. The invention provides a method to examine a given cell for morphological/histological abnormalities, and either sequentially or simultaneously examine the same cell for nucleic acid targets indicative of HPV infection. Together, the combined tests have positive and negative predictive values equal to or greater than either test alone.

In other embodiments, the invention provides high specificity of test results by detecting active expression of high risk HPV types directly in abnormal cells, particularly those abnormal cells found in cervical specimens. Unlike current methods, individual abnormal cells observed by Pap staining can be linked with an active HPV infection of high risk types and thus potentially eliminate clinically insignificant HPV results. This has particular importance for women with equivocal or borderline abnormal Pap test results known as ASCUS where HPV testing is recommended to determine the likelihood for underlying precancerous or cancerous changes in the cervical tissue.

In another embodiment, the presence of the HPV mRNA is indicative of the presence of a particular HPV type. In related embodiments, the HPV mRNA is indicative of the presence of HPV strains selected from types 16, 18, 31,33,35,39,45,51,52, 56,58,59,68 and 70.

In another preferred embodiment, methods and high-affinity probes are provided for detection of HPV messenger RNA. The messenger RNA comprising any of the HPV open reading frames or splice variants thereof, including E6, E7, and E6-E7.

In still another embodiment, this invention is directed towards kits suitable for performing an assay that detects expression of high risk HPV types in cervical specimens by in situ hybridization and fluorescent microscopy. The kits of this invention comprise one or more high affinity probes and other reagents or compositions that are selected to perform an assay or otherwise simplify the performance of an assay.

Methods and kits of the invention comprise detecting molecular markers of a disease state. Molecular markers include cancer marker genes and their corresponding mRNA transcripts such as the Brn-3a Bcl-2, Her2-neu, p53, E6/E7, E1/E2 or a growth factor receptor genes.

In one embodiment, methods are used to detect nucleic acids indicative of HPV infection, which in turn provides a basis for medical diagnosis and treatment. Methods of the invention advance the current state of the art by increasing the speed and specificity of detection and by increasing the prognostic value of the test. Thus, it is an object of the present invention to provide a method for assessing the stage of HPV-based disease.

It is another object of the present invention to provide an assay that can be combined with other assays to improve the accuracy and reliability of prognostic and diagnostic assessments of HPV-based disease. Another object of the invention is to provide a method for monitoring the effectiveness of treatment of HPV-based disease. A further object of the invention is to provide kits for assessing the stage of HPV-based disease. These and other advantages and aspects of the invention will be understood upon consideration of the following detailed description thereof.

### BRIEF DESCRIPTION OF FIGURES

FIG. 1 shows fluorescent image of GAPDH probed and control cells at 600× magnification.

### DETAILED DESCRIPTION

1. Definitions:

a. As used herein, the term “nucleobase” means those naturally occurring and those non-naturally occurring heterocyclic moieties commonly known to those who utilize conventional nucleic acid technology or utilize locked nucleic acid or peptide nucleic acid technology to thereby generate polymers that can sequence specifically bind to nucleic acids.

b. As used herein, the term “nucleobase sequence” means any segment of a polymer that comprises nucleobase-containing subunits. Non-limiting examples of suitable polymers or polymer segments include deoxynucleic acid (DNA), oligodeoxynucleotides, deoxyribonucleic acid (RNA), oligoribonucleotides, locked nucleic acids (LNA), peptide nucleic acids (PNA), nucleic acid analogs, nucleic acid mimics, and/or chimeras.

c. As used herein, the term “target sequence” means the nucleobase sequence that is to be detected in an assay. Target sequences may be, for example, genomic DNA, RNA, mRNA (e.g., spliced or nascent message), mitochondrial DNA or RNA, and the like. Spliced, as used herein, refers to the processing of a nascent RNA message.

d. As used herein, the term “probe” means a polymer (e. g. a DNA, RNA, PNA, LNA, chimera or linked polymer) having a probing nucleobase sequence that is designed to sequence-specifically hybridize to a target sequence of an microorganism of interest.

e. As used herein, “analyze” means that probe-specific nucleic acid target sequences are marked for detection, typing and/or quantitation.

f. As used herein, “expression” means transcription of a DNA template by a polymerase to generate a reverse complement comprised primarily of a ribonucleic acid polymer.

g. As used herein, “mRNA” means any ribonucleic acid transcribed from an open reading frame of a DNA template. mRNA includes those ribonucleic acid transcripts which have and have not been post-transcriptionally processed. Post-transcriptional processing includes, but is not limited to splicing, trans-splicing, intron excision, 5′ capping, 3′cleavage, and polyadenylation.

h. As used herein, the term “high affinity probe” means a probe with improved hybridization kinetics as compared to conventional deoxyribonucleic acid (DNA) probes. High affinity probes of the invention may be made of the polymers described herein.

i. As used herein, the term “peptide nucleic acid” or “PNA” means any oligomer, linked polymer or chimeric oligomer, comprising two or more PNA subunits (residues), including any of the polymers referred to or claimed as peptide nucleic acids in U.S. Pat. Nos. 5,539,082, 5,527,675, 5,623,049, 5,714,331, 5,736,336, 5,773,571, 5,786,461, 5,837,459, 5,891,625, 5,972,610, 5,986,053, 6,107,470 and 6,357,163. In a preferred embodiment, a PNA subunit consists of a naturally occurring or non-naturally occurring nucleobase attached to the aza nitrogen of the N-[2-(aminoethyl)]glycine backbone through a methylene carbonyl linkage.

j. As used herein, the term “locked nucleic acid” or “LNA” means any oligomer, linked polymer or chimeric oligomer, comprising one or more LNA subunits (residues), including any of the polymers referred to or claimed as locked nucleic acids in U.S. Pat. Nos. 6,794,499 and 6,670,461, which are hereby incorporated by reference in their entirety. In one embodiment, an LNA subunit consists of a nucleotide analogue as described in U.S. Pat. Nos. 6,794,499 and 6,670,461.

k. As used herein, the terms “label” and “detectable moiety” are interchangeable and shall refer to moieties that can be attached to a probe to thereby render the probe detectable by an instrument or method.

l. Percent (%) identity, with respect to two nucleic sequences, refers to the percent of nucleic acids that are identical in the two sequences when the sequences are optimally aligned. Optimal alignment is defined as the alignment giving the highest percent identity score. Such alignments can be performed using the “GENEWORKS” program. Alternatively, alignments may be performed using the local alignment program LALIGN with a ktup of 1, default parameters and the default PAM. See also http://www.ncbi.nim.nih.gov. In the context of the present invention, when it is stated that a probe has 80% identity to a given sequence, it is implicit that this refers to the entire sequence of the longer of the two probes. Substantially identical, as used herein includes probes sequences that share 90% or greater identity and are functionally identical.

2. Description

Nucleic acid hybridization is a fundamental physiochemical process, central to the understanding of molecular biology. Probe-based assays use hybridization for the detection, quantitation and analysis of nucleic acids. Nucleic acid probes have long been used to analyze samples from a variety of sources for the presence of nucleic acids, as well as to examine clinical conditions of interest in single cells and tissues. More recently, high affinity nucleic acid probe analogs and mimics have become the preferred reagents for hybridization assays.

Fundamental to the understanding of nucleic acid probes is the understanding of hybrid melting temperatures (Tm). The Tm of a probe-target hybrid is an idealized equilibrium, defined as the temperature at which 50% of the probes are hybridized, and 50% are non-hybridized. This equilibrium is dependant on several factors including salt concentration, probe concentration, target concentration, and pH.

Generally, hybridization assays are designed to achieve high specificity, meaning that probes only hybridize to perfectly matched (fully complementary), or nearly perfectly matched (partially complementary) targets. Many technologies have been developed to aid in achieving high specificity. For example, denaturants such as formamide, urea, or formaldehyde can be used to lower the effective Tm of a probe. Chaotropic salts such as guanidinium thiocyanate, tetramethylammonium chloride, guanidinium hydrochloride, sodium thiocyanate and others used at high concentrations disrupt the formation of hydrogen bonds. Manipulations of the factors defined by Tm such as temperature, or probe concentration directly affect the specificity of probes. Other factors such as competition with other probes, or use of blocker probes (see U.S. Pat. No. 6,110,676) will also affect the specificity.

This equilibrium is dependant on several factors including salt concentration, probe concentration, target concentration, and pH (except for HA probes)

Locked Nucleic Acid (LNA) and Peptide Nucleic Acid (PNA) are novel high affinity probes which provide higher sensitivity and specificity than conventional DNA probes. DNA is a biological material that plays a central role in the life of living species as the agent of genetic transmission and expression, LNA and PNA are recently developed totally artificial molecules, conceived in the minds of chemists and made using synthetic organic chemistry. Although LNA and PNA can employ common nucleobases (A, C, G, T, and U) and can hybridize to nucleic acids with sequence specificity according to Watson-Crick base paring rules, they differ both structurally and functionally from DNA., Peptide Nucleic Acid, despite its name, is neither a peptide nor a nucleic acid, nor is it even an acid, but a non-naturally occurring polyamide backbone composed of (aminoethyl)-glycine subunits where the nucleobases are connected to the backbone by an additional methylene carbonyl moiety. (See: U.S. Pat. No. 5,539,082) and Egholm et al., Nature 365:566-568 (1993)). Due mostly to the fact that PNA carries a net neutral electrical charge, PNA can form hybrids extremely rapidly and stably with naturally occurring nucleic acids. LNA is a nucleic acid analog created by chemically joining the 2′ oxygen and 4′ carbon of a ribonucleoside through a methylene linkage. The highly rigid structure of the resultant locked 3′-endo conformation reduces the conformational flexibility of the ribose. The increased rigidity and local organization of the LNA phosphate backbone lowers the entropic penalty for hybridization, increasing the Tm of LNA probes as compared to DNAs of the same relative composition. These structural features provide PNA and LNA probes with higher affinity for target sequences and furthermore allow PNA and LNA probes to hybridize under conditions that are destabilizing to naturally occurring nucleic acids, such as low salt concentration or in the presence of guanidinium hydrochloride. These attributes enable PNA probes to access targets, such as highly structured rRNA and double stranded DNA, known to be inaccessible to DNA probes (See: Stefano & Hyldig-Nielsen, IBC Library Series Publication #948. International Business Communication, Southborough, MA, pp.19-37 (1997), (Fuchs, Appl Envir Micro 64 (12) 4973-82, 1998; Singh, Chem. Commun. 4 455-456, 1998)). LNA and PNA are useful candidates for investigation when developing novel probe-based hybridization assays because of their excellent hybridization features.

Fluorescent in situ hybridization (FISH) is a widely used technique for direct fluorescent visualization of biological molecules maintained within cellular structures. FISH experiments can be performed on any cell type, and can be used to detect any type of target molecule. Examples of FISH technologies using high affinity probes include chromosomal analysis (Silahtaroglu, Mol. Cell. Probes, (17) 165-9, 2003; Taneja, Biotechniques (24) 472-6,1998), identification of microorganisms (Perry-O'Keefe, J. Micro. Meth. (47) 281-292, 2001), and flow cytometry (Xi, Appl Env Microbiol 2003. 69(9) 5673-8). Typically cells and their components are chemically preserved using fixatives such as formamide, paraformaldehyde, gluteraldehyde, formallin, formaldehyde, methanol, ethanol, or paraffin including commercial fixatives such as PreservCyt (Cytyc, Marlboro Mass.) or SurePath (Tripath, Burlington, N.C.). The benefit of FISH techniques is that they preserve the molecular targets at metabolic (high) concentrations. For example, a bacterium with a volume of 1 μm³ can contain 10,000 ribosomal RNAs, a concentration of approximately 15 uM (Loferer-KröβBacher, Appl Env Microbiol 1998. 64(2):688-694). The effect relative to all other methods of molecular probing is an extremely high concentration of target molecules within the minute spaces of cells, obviating the need for amplification steps. Combined with microscopy, the effect is a relatively easy detection of targets preserved in intact cellular morphologies. In situ analysis does not necessitate use of a microscope, for instance confirmation of bacterial cell cultures has been performed on an array scanner (Stender, J. Micro. Meth. (45) 31-39, 2001) and could conceivably be performed in a microtiter plate and measured on a fluorescent plate reader.

I. General:

PNA Synthesis:

Methods for the chemical assembly of PNAs are well known (see: U.S. Pat. Nos. 5,539,082, 5,527,675, 5,623,049, 5,714,331, 5,736,336, 5,773,571, 5,786,461, 5,837,459, 5,891,625, 5,972,610, 5,986,053 and 6,107,470, which are hereby incorporated by reference in their entireties).

LNA Synthesis:

Methods for the chemical assembly of LNAs are well known (see: Patent Nos. U.S. Pat. Nos. 6,794,499 and 6,670,461.)

PNA Labeling:

Preferred non-limiting methods for labeling PNAs are described in U.S. Pat. No. 6,110,676, 6,361,942, 6,355,421, the examples section of this specification or are otherwise well known in the art of PNA synthesis and peptide synthesis.

LNA Labeling:

Preferred non-limiting methods for labeling LNAs are described in US, the examples section of this specification or are otherwise well known in the art of PNA synthesis and peptide synthesis.

One of skill in the art, having the benefit of this disclosure would understand how to assemble and label the PNA and LNA probes of the invention.

Labels:

Non-limiting examples of detectable moieties (labels) suitable for labeling PNA probes used in the practice of this invention would include a dextran conjugate, a branched nucleic acid detection system, a chromophore, a fluorophore, a spin label, a radioisotope, an enzyme, a hapten, an acridinium ester and a chemiluminescent compound. Other suitable labeling reagents and preferred methods of attachment would be recognized by those of ordinary skill in the art of PNA, peptide, LNA or nucleic acid synthesis. Haptens include 5 (6)-carboxyfluorescein, 2,4-dinitrophenyl, digoxigenin, and biotin. Fluorochromes (fluorophores) include 5 (6)-carboxyfluorescein (Flu), tetramethyl-6-carboxyrhodamine (tamra), 6-((7-amino-4-methylcoumarin-3-acetyl)amino)hexanoic acid (Cou), 5 (and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2,3,3.5,5 and 5.5 are available as NHS esters from Amersham, Arlington Heights, Ill.), JOE, Tamara or the Alexa dye series (Molecular Probes, Eugene, Oreg.). Enzymes include polymerases (e.g. Taq polymerase, Klenow PNA polymerase, T7 DNA polymerase, Sequenase, DNA polymerase 1 and phi29 polymerase), alkaline phosphatase (AP), horseradish peroxidase (HRP) and most preferably, soy bean peroxidase (SBP).

Self-Indicating Probes:

Self indicating probed include Beacon probes and of the type described in WIPO patent application WO97/45539. Beacon probes include a donor moiety and a acceptor moiety. The donor and acceptor moieties operate such that the acceptor moieties accept energy transferred from the donor moieties or otherwise quench signal from the donor moiety. Though the previously listed fluorophores (with suitable spectral properties) might also operate as energy transfer acceptors, preferably, the acceptor moiety is a quencher moiety. Preferably, the quencher moiety is a non-fluorescent aromatic or heteroaromatic moiety. The preferred quencher moiety is 4-((-4-(dimethylamino)phenyl)azo)benzoic acid(dabcyl). In a preferred embodiment, the self-indicating Beacon probe is a PNA Linear Beacon as more fully described in U.S. Pat. No. 6,485,901.

The self-indicating PNA probes of the type described in WIPO patent application WO97/45539 differ as compared with Beacon probes primarily in that the reporter must interact with the nucleic acid to produce signal.

Spacer/Linker Moieties:

Generally, spacers are used to minimize the adverse effects that bulky labeling reagents might have on hybridization properties of probes. Preferred spacer/linker moieties for the nucleobase polymers of this invention include of one or more aminoalkyl carboxylic acids (e.g. aminocaproic acid), the side chain of an amino acid (e.g. the side chain of lysine or omithine), natural amino acids (e.g. glycine), aminooxyalkylacids (e.g. 8-amino-3,6-dioxaoctanoic acid), alkyl diacids (e.g. succinic acid), alkyloxy diacids (e.g. diglycolic acid) or alkyldiamines (e.g. 1,8-diamino-3,6-dioxaoctane). A linker or spacer of the invention may also be made up of linker units selected from units of formulas —NH—(CH₂CH₂O)ₙCH₂C(O)—, —NH(CHOH)ₙC(O)—, —(O)C(CH₂OCH₂)ₙC(O)— and —NH(CH₂)ₙC(O)—, wherein n is 0 or an integer from 1 to 8, preferably from 1 to 3. A linker unit may have a free amino group or a free acid group, i.e. NH₂(CH₂CH₂O)ₙCH₂C(O)—, NH₂(CHOH)N C(O)—, HO(O)C(CH₂OCH₂)ₙC(O)—, NH₂(CH₂)ₙC(O)—, —NH(CH₂CH₂O)ₙCH₂C(O)OH, —NH(CHOH)ₙC(O)OH, —(O)C(CH₂OCH₂)ₙC(O)OH and —NH(CH2)ₙC(O)OH. A linker may consist of up to 3 of such linker units. Examples interesting linker units are —NHCH₂C(O)—, —NHCH₂CH₂C(O)—, —NH(CH₂CH₂O)₂CH₂C(O)—, HO(O)CCH₂C(O)(NH—(CH₂CH₂O)₂CH₂C(O))₂—.

Hybridization Conditions/Stringency:

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well known technique of fixing several of the aforementioned stringency factors and then determining the effect of varying a single stringency factor. The same stringency factors can be modulated to thereby control the stringency of hybridization of high affinity probes to a nucleic acid, although high affinity probes do differ in their functions and for example the hybridization of a PNA is fairly independent of ionic strength. Optimal stringency for an assay may be experimentally determined by examination of each stringency factor until the desired degree of discrimination is achieved.

A method for expression analysis of human papillomavirus comprising contacting a cytological specimen with a high affinity probe of any one of claims 1-12 under conditions suitable for in situ hybridization. In certain methods, the in situ hybridization is fluorescence in situ hybridization. Two or more high affinity probes may be used on the same sample and these probes may be are similarly labeled or differently labeled. Techniques for labeling and detection of similarly or differentially labeled probed are described herein. The probes useful in the invention include a mixture of type-specific HPV probes.

HPV-based disease, as used herein, refers to diseases caused or exacerbated by an HPV infection. Diseases include, for example, genital warts and benign tumors, which are generally of three symtomologic classes, gential-mucosal, non-genital and epidermiodysplasia verruciformis (EV)-specific epidermiodysplasia verruciformis (EV)-specific.

The methods herein provide an approach to identify or assess the stage of an HPV-based disease. The assessment may be by a comparison or correlation to an earlier assessment or may be based on a comparison with a control or reference. The control or reference may be a standard indicating that certain ranges of level are correlative with a certain disease state. For example, a very high level of HPV from multiple samples from the same subject taken from different locations may indicate an advanced stage of disease. A decrease in the level of HPV over time may indicate that treatment or healing is taking place.

The methods further comprise a second, third, fourth, or more assays after the initial assay. The initial assay may provide a base-line for comparison or correlation purposes. The second assay may be a repeat of the first assay or may be a different assay, for example, Pap staining. Alternatively the invention described may be part of an assay which is performed after an initial diagnostic method or methods.

Furthermore, a second assay may be performed simultaneously with the first. The second assay may be a histological staining assay such as the Pap stain which could be performed using a combined reagent. Since histological staining and in situ hybridization methods are both performed on glass slides combining the methods to produce both assay results only requires finding appropriately compatible reagents. Where reagent incompatibility prevents combined assays, assays may still be performed in succession such that both results from both assays are available at once, and may be inspected simultaneously.

Methods of assessing the risk of developing HPV-based disease are also provided and comprise contacting a sample from a subject with one or more high affinity probes complementary to a target sequence of human papillomavirus mRNA, and determining the presence of bound probe. Unbound probe can be removed in washing steps described herein. Proves useful are probes identified by SEQ ID NOS: 1-14 or derivatives thereof. The determining the presence of bound probe may be by in situ hybridization.

The methods may further comprise correlating the presence or absence of papillomavirus mRNA with a risk of developing HPV-based disease. The correlating may, for example, distinguished between level of risk. Levels of risk include no risk, moderate risk, and high risk. No risk correlates with no detectable HPV. Moderate risk correlates with detection an HPV infection but no detectable HPV-based disease. High risk correlates with the detection of high levels of HPV infection.

Methods are provided for selecting subjects for treatment for HPV-based diseases. The methods comprise contacting a sample from a subject with one or more high affinity probes complementary to a target sequence of human papillomavirus mRNA, determining the presence of bound probe, and correlating the presence of papillomavirus mRNA with a need for treatment for HPV-based diseases. The methods may further comprise treating a subject for HPV-based diseases based on the presence of papillomavirus mRNA in the selection assay. Treatment options may be based on the level of the virus.

Method for monitoring the efficacy of an HPV-treatment comprise determining a pre-treatment level of HPV infection, administering an HPV infection treatment, and determining a post-treatment level of HPV infection after an initial period of treatment the initial period of treatment is the time required to achieve a steady-state plasma or cellular concentration of an HPV-based treatment. Treatments may include, chemotherapy, anti-viral therapy, radiation therapy, and the like. A decrease in the level of infection is an indication that the treatment is efficacious.

The pre-treatment and post-treatment levels of HPV infection are determined by contacting a sample from a subject with one or more high affinity probes complementary to a target sequence of human papillomavirus mRNA and detecting the presence of bound probe.

Methods for the expression analysis of human papillomavirus by in situ hybridization, comprise contacting the sample with at least one high-affinity probe that is substantially complementary to a portion of a HPV mRNA; incubating the sample with the high affinity probe; and detecting the fluorescence of the sample, wherein the level of fluorescence is indicative of the presence and/or amount of mRNA within individual cell of the sample. The presence of the HPV may be by the detection of the expressed mRNA. The methods may further comprise correlating a level of fluorescence with a down-regulation of expression of HPV mRNA. A decrease in fluorescence is correlative with down-regulation and an increase in fluorescence is correlative of up regulation of expression of HPV mRNA. Comparisons of the methods may be of the expression of two or more mRNAs. For example, one or more of Bcl-2, Her2-neu, p53, E6/E7, E1/E2 or genes for growth factor receptors.

The invention also provides methods of diagnosing or predicting an HPV-based disease in a subject, comprising determining a level of HPV infection by contacting a sample from a subject with one or more high affinity probes complementary to a target sequence of human papillomavirus mRNA, and determining the presence of bound probe; comparing the level, to a standard level; and correlating a modulated level in the cell from the subject with an indication of an HPV-based disease. The standard level is the corresponding level in a reference cell or population of cells. The reference cell is one or more of the following, cells from the subject, cultured cells, cultured cells from the subject, cells from the subject pre-treatment, cells from a second subject not suspected or showing no signs of an HPV-based disease. The methods may further comprise obtaining a cell sample from the subject and/or reporting the level or correlations thereof to the subject or a health care professional.

In one embodiment, high affinity probes of the invention are also useful for the detection of or expression analysis of cancer marker genes. For example, for the detection of cancer marker gene is Brn-3a. Related methods include expression analysis of a cancer marker genes comprising contacting a cytological specimen with a high affinity probe under conditions suitable for in situ hybridization. Two or more high affinity probes may be used on the same sample and these probes may be are similarly labeled or differently labeled. Techniques for labeling and detection of similarly or differentially labeled probed are described herein.

Generally, the more closely related the background causing nucleic acid contaminates are to the target sequence, the more carefully stringency must be controlled. Blocking probes may also be used as a means to improve discrimination beyond the limits possible by optimization of stringency factors. Suitable hybridization conditions will thus comprise conditions under which the desired degree of discrimination is achieved such that an assay generates an accurate (within the tolerance desired for the assay) and reproducible result.

Aided by no more than routine experimentation and the disclosure provided herein, those of skill in the art will easily be able to determine suitable hybridization conditions for performing assays utilizing the methods and compositions described herein. Suitable in-situ hybridization comprises conditions suitable for performing in-situ hybridization procedures. Thus, suitable in-situ hybridization conditions will become apparent to those of skill in the art using the disclosure provided herein, with or without additional routine experimentation.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ° C. to about 20° C. to 25° C. below Tₘ. As will be understood by those of skill in the art, a stringent hybridization can be used to identify or detect identical polynucleotide sequences or to identify or detect similar or related polynucleotide sequences. Under “stringent conditions” the probes of the invention or portions thereof will hybridize to its exact complement and closely related sequences and thus, for example, differentiate between HPV types.

Hybridizations can be performed using fixed, immobilized or suspended sample preparations, (e.g., tissue slices of biopsy material and cells in suspension from a sample obtained by swabbing or scraping). If a double-stranded target such as chromosomal or DNA sequences are to be detected, a treatment to separate the two strands may be used. This separation of the strands can be achieved by heating the sample in the presence of the hybridization mixture to a temperature sufficiently high and for a time period sufficiently long to dissociate the strands. Typically, heating at a temperature of 90° C. to 95° C. for a period of 5 to 15 minutes is suitable.

Hybridization buffers may comprise a hybrid destabilizing agent in an amount effective to decrease the melting temperature of hybrids formed between the nucleic acid to be determined and the probe so as to increase the ratio between specific binding and non-specific binding. This agent will allow the hybridization to take place at a lower temperature than without the agent. In traditional nucleic acid hybridization, such agent is called a denaturing agent.

Hybridization and denaturing may be carried out simultaneously using a suitable amount a hybrid destabilizing agent in combination with a suitable temperature for the treatment.

The amount of the hybrid destabilizing agent used will depend on the type of destabilizing agent and on the probe or combination of probes. For example, hybrid destabilizing are useful in the invention and include formamide, ethylene glycol and glycerol and these agents can be used in a concentration above 10% and less than 70%. The concentration of formamide may be from about 20% to about 60%, preferably from 30% to 50%. The concentration of ethylene glycol may also be from 30% to 65%, and preferably a concentration of 65%. The concentration of glycerol may more preferably be from 45% to 60%, most preferably 50%.

It may be advantageous to include macromolecules or polymers such as dextran sulphate, polyvinylpyrrolidone and/or ficoll. In the presence of such macromolecules or polymers, the effective concentration of the probe at the target is assumed to be increased. Dextran sulphate may be added in a concentration of up to 15%. Concentrations of dextran sulphate of from 2.5% to 12.5% are often advantageous.

In some instances, it may be advantageous to add a detergent such as sodium dodecyl sulphate, Tween 20 or Triton X-100.

During hybridization, other important parameters are hybridization temperature, concentration of the probe and hybridization time. The person skilled in the art will readily recognize that optimal conditions for various starting materials will have to be determined for each of the above-mentioned parameters.

Post-Hybridization Washing

Following hybridization, the preparation is washed to remove any unbound and any non-specifically bound probes. The conditions described below are merely by way of example and may depend on the type of preparation to be analyzed. During the post-hybridization step, appropriate stringency conditions should be used in order to remove any non-specifically bound probe. Stringency refers to the degree to which reaction conditions favor the dissociation of the formed hybrids and may be enhanced, for instance by increasing the washing temperature and incubation time. For conventional hybridization with nucleic acid probes, the salt concentration is often used as an additional factor for regulating the stringency. This may not apply to probes comprising polymerized LNA and/or PNA moieties as the binding of this type of probes has been shown to be virtually independent of the salt concentration (Nature, 365, 566-568 (1993)).

Examples of useful buffer systems are Tris-Buffered-Saline (TBS), standard citrate buffer (SSC) or phosphate buffers. A convenient TBS buffer is 0.05M Tris/HCl, 0.15M NaCI, pH 7.6. The SSC buffer comprises 0.15M sodium chloride and O.015M trisodium citrate, pH 7.0.

Typically, washing times from 25 to 30 minutes are suitable. Washing periods of two times 10 minutes or 3 times 5 minutes in a suitable buffer are also suitable.

In some cases, particularly when using probes carrying at least one fluorescein label, it has been shown to be advantageous to increase the pH of the washing buffer. An increase in the signal-to-noise ratio has been observed using a washing buffer with an alkaline pH. This is apparently due to a significantly reduction of the non-specific binding. In such cases, it is preferred that the washing solution in step (3) has a pH value of from 8 to 10.5, preferably from 9 to 10.

Detection

In cases where the sample is deposited onto slides, the hybridization results may be visualized using well known immunohistochemical staining methods to detect the labeled probe. When fluorescent labeled probes are used, the hybrids may be detected using an antibody against the fluorescent label which antibody may be conjugated with an enzyme. The fluorescent label may alternatively be detected directly using a fluorescence microscope, or the results may be automatically analyzed on a fluorescent-based image analysis system.

When biotin labeled probes are used, the hybrids may be detected using an antibody against the biotin label which antibody may be conjugated with an enzyme. If necessary, an enhancement of the signal can be generated using commercially available amplification systems such as the catalyzed signal amplification system for biotinylated probes (DAKO K 1500).

In the case of a suspended sample such as a cell suspension, quantitative results may be obtained using probes comprising a fluorescent label and a flow cytometer to record the intensity of fluorescence per cell.

Probes used in some aspects of the present method may form nucleic acid/probe hybrids which can be recognized by an antibody described in WO 95/17430. Hybrids formed between the probe, nucleic acid and the antibody can be detected in a direct immunohistochemical staining method using, for instance an enzyme conjugated form of the antibody, followed by detection of the enzyme activity or by the application of well known indirect immunohistochemical staining techniques.

Blocking Probes:

Blocking probes are nucleic acid or non-nucleic acid probes that can be used to suppress the binding of the probing nucleobase sequence of the probing polymer to a non-target sequence. Preferred blocking probes are PNA probes (see: U.S. Pat. No. 6,110,676). Without wishing to be bound by any theories, it is believed that blocking probes operate by hybridization to the non-target sequence to thereby form a more thermodynamically stable complex than is formed by hybridization between the probing nucleobase sequence and the non-target sequence. Formation of the more stable and preferred complex blocks formation of the less stable non-preferred complex between the probing nucleobase sequence and the non-target sequence. Thus, blocking probes can be used with the methods, kits and compositions of this invention to suppress the binding of the probes to a non-target sequence that might be present and interfere with the performance of the assay.

A further benefit of using high affinity probes for detection of mRNA in situ is through simplification of the assay by omission of unlabeled probes to prevent non-specific binding. WO03027328 and U.S. Pat. No. 5,776,688 both supplied herein by reference, teach that preferred methods of performing in situ hybridization experiments to detect chromosomal targets require non-labeled probes to limit non-specific binding to non-target sequences, particularly repeat sequences. Since high affinity probes are highly specific this requirement is obviated.

Probing Nucleobase Sequence:

The probing nucleobase sequence of a probe of this invention is the specific sequence recognition portion of the construct. Therefore, the probing nucleobase sequence is a nucleobase sequence designed to hybridize to a specific target sequence wherein the presence, absence or amount of the target sequence can be used to directly or indirectly detect the presence, absence or number of organisms of interest in a sample. Consequently, with due consideration to the requirements of a probe for the assay format chosen, the length and sequence composition of the probing nucleobase sequence of the probe will generally be chosen such that a stable complex is formed with the target sequence under suitable hybridization conditions, for example, stringent hybridization conditions.

Examples of probing nucleobase sequences of the probes of this invention that are suitable for analysis of expression of HPV types are listed in Table 1.

TABLE 1

Probing nucleobase sequences targeting
spliced E6 and E7 of various ‘high risk’
HPV types.

Probing nucleobase

| | | |
|---|---|---|
| Seq. Id. Number | Probe Name | sequence |

| | | |
|---|---|---|
| Seq. Id. No. 1 | HPV16 E6 | ATA TAC CTC ACG TCG |
| Seq. Id. No. 2 | HPV18 E6 | AGG CAC CTC TGT AAG |
| Seq. Id. No. 3 | HPV31 E6 | ATA CAC CTC TGT TTC |
| Seq. Id. No. 4 | HPV33 E6 | ATA CAC CTC AGA TCG |
| Seq. Id. No. 5 | HPV35 E6 | ATA CAC CTC ACT CCG |
| Seq. Id. No. 6 | HPV18 E7 | GTC TTC CAA AGT ACG |
| | | A |
| Seq. Id. No. 7 | HPV35 E7 | TTC CAA TTT ACG TAT |
| | | GTC |
| Seq. Id. No. 8 | HPV16 E7 | TCC AAA GTA CGA ATG |
| | | T |
| Seq. Id. No. 9 | HPV33 E7 | GTA TGG TTC GTA GGT |
| Seq. Id. No. 10 | HPV31 E7 | CTT GCA ATA TGC GAA |
| | | TAT |
| Seq. Id. No. 11 | HPV16 E6 B | TCG CAG TAA CTG TTG |
| | | C |
| Seq. Id. No. 12 | HPV16 E6 C | TCA CGT CGC AGT AAC |
| Seq. Id. No. 13 | 16E6E7 A | TAA TAC ACC TCA CGT |
| Seq. Id. No. 14 | 16E6E7 B | GTT AAT ACA CCT CAC |

This invention contemplates that variations in these identified probing nucleobase sequences shall also provide probes that are suitable for the analysis of expression of HPV. Variation of the probing nucleobase sequences within the parameters described herein are considered to be an embodiment of this invention. Common variations include, deletions, insertions and frame shifts are included within the scope of the invention. Additionally, a shorter probing nucleobase sequence can be generated by truncation of the sequence identified above.

A probe of this invention will generally have a probing nucleobase sequence that is exactly complementary to the target sequence. Alternatively, a substantially identical probing nucleobase sequence might be used since it has been demonstrated that greater sequence discrimination can be obtained when utilizing probes wherein there exists one or more point mutations (base mismatch) between the probe and the target sequence (See: Guo et al., Nature Biotechnology 15: 331-335 (1997)). Consequently, the probing nucleobase sequence may be only 90% identical to the probing nucleobase sequences identified above. Substantially identical probing nucleobase sequence within the parameters described above are considered to be an embodiment of this invention.

Probes, kits and methods of this invention are further directed toward detection of mRNA sequences of particular genes which may also be implicated in cancer or other disease state. Up or down regulation of gene expression is commonly described in the literature as indicative of a disease state. Selection of probes for other targets not described here, but understood to be of clinical value follows the same method as described herein for design of high-affinity probes directed against HPV mRNA, with the exception of the target sequence. For example, probes may be selected for cancer markers, including Brn-3a, Bcl-2, Her2-neu, p53, E6/E7, E1/E2 or a growth factor receptor. The sequences of these genes are known as accessed by one of skill in the art, for example, at GENBANK, http://www.ncbi.nlm.nih.gov/entrez/query. fcgi?db=Nucleotide. Accession numbers for selected cancer markers include, Brn-3a (POU4F1), ACCESSION # NM_{—}006237; Bcl-2, ACCESSION # M14745; Her2-neu (ERBB2), ACCESSION # NM_{—}004448; p53, ACCESSION # NM_{—}000546; and E1, E2, E6, E7, ACCESSION # NC_{—}001526.

Probe for mRNA or specifically for cancer markers may be, for example, from 8-20 in length and directed to unique portions of the gene or mRNA.

Samples, as used herein, include blood, biopsies of epithelial cells or cells from swabbing or scraping of a tissue, for example, uterine cervical tissue. The sample to be examined is pre-treated before the hybridization step whereby a preparation of the sample is produced. A person skilled in the art will readily recognize that the appropriate pretreatment will depend on the type of sample to be examined. During the pretreatment, the sample will be subject to a fixation.

In one embodiment of the method, the sample is deposited onto a solid support. Techniques for depositing a sample onto the solid support will depend upon the type of sample in question and may include, for example, sectioning of tissue as well as smearing or cytocentrifugation of cell suspensions. Many types of solid supports may be utilized to practice the present method. The use of such supports and the procedures for depositing samples thereon are known to those skilled in the art. Glass microscope slides are especially convenient. Glass microscope slides can be treated to better retain the sample.

Prior to hybridization, the sample is suitably pre-treated with various chemicals to facilitate the subsequent reactions. The actual pretreatment will depend on the type of sample to be analyzed and on whether DNA or RNA sequences are to be detected. For localizing RNA such as mRNA, it is advantageous that the sample is treated as soon as possible after sample collection to retain most of the RNA intact.

In producing a preparation of a tissue sample, the morphological integrity of a tissue and the integrity of the nucleic acids can be preserved by bringing the sample to a fixed stage either by means of chemical fixation or freezing. When freezing is used for preservation of for instance a biopsy, the biopsy is typically frozen in liquid nitrogen. After freezing, the sample may appropriately be stored at −80° C. Prior to the analysis of the nucleic acid, the frozen sample is cut into thin sections and transferred to e.g. pre-treated slides. This can e.g. be carried out at a temperature of −20° C. in a cryostat. The biopsy or tissue sections may suitably be stored at −80° C. until use. Prior to hybridization, the tissue section may be treated with a fixative, preferably a precipitating fixative such as acetone or the tissue section is incubated for a short period in a solution of buffered formaldehyde. Alternatively, the biopsy or tissue section can be transferred to a fixative such as buffered formaldehyde for 12 to 24 hours. Following fixation, the tissue may be embedded in paraffin forming a block from which thin sections can be cut. Well prepared paraffin-embedded samples can be stored at room temperature for a period of years.

Prior to hybridization, the tissue section is dewaxed and rehydrated using standard procedures.

Further permeabilization may be necessary in order to ensure sufficient accessibility of the target nucleic acid sequences to the probe. The type of treatment will depend on several factors, for instance on the fixative used, the extent of fixation, the type and size of sample used and the length of the probe. The treatment may involve exposure to protease such as proteinase K, pronase or pepsin, diluted acids, detergents or alcohols or a heat treatment.

For analyzing a suspended preparation such as a suspension of cells, the sample is treated so as to obtain a permeabilization of the material and a preservation of the morphology. Fixation may be carried out with a fixative such as formaldehyde, acetone or ethanol.

In Situ Hybridization Techniques in Tissue Sections

A biopsy is treated to preserve the morphological integrity of the cellular matrix and of the nucleic acid within the cell. The biopsy is brought to a fixed stage either by means of a chemical fixation or by freezing, e.g., formaldehyde, preferably as a 4% v/v solution in buffer at neutral pH. After fixation for typically from 12 to 14 hours, the biopsy is embedded in paraffin. The paraffin embedded biopsy may be used immediately or may be stored at room temperature for a period of years. From the paraffin embedded biopsy, thin sections having a thickness of typically 3-6 μm are cut and transferred onto silanized or otherwise adhesive-treated microscope slides.

The slide may then be dried, for instance by incubating the slide for 30 minutes at 60° C.

The slides are dewaxed, for instance by immersion in a dewaxing solution such as xylene, and rehydrated, e.g. by immersion in 99% ethanol, 95% ethanol, air-drying and immersion into for instance Milli Q water. To increase the accessibility of the target sequences to the probe, the tissue section may be treated with a proteolytic agent such as proteinase K. The slides are rinsed in a suitable buffer such as a TBS-buffer.

High affinity probes, capable of forming sufficiently stable hybrids with mRNA are selected and synthesized.

An appropriate amount of unlabelled or labeled probe is brought in contact with the tissue section together with an appropriate hybridization mixture comprising a hybrid destabilizing agent. In a preferred embodiment, the hybridization mixture comprises from 30% to 50% formamide. The tissue section on the slide is incubated at an appropriate temperature for an appropriate period of time. Typically, a probe concentration of from 5 to 1000 nM, incubation temperatures of from 40° C. to 60° C. and hybridization times of from 10 to 120 minutes are used.

The slides may then be washed to remove any unbound and any non-specifically bound probe. The slides are typically washed in a TBS-buffer at a temperature of from 40° C. to 65° C. for from 15 to 45 minutes. Non-specific binding may be reduced significantly using an alkaline washing buffer. A washing buffer having a pH value from 8 to 10.5 may be employed, preferably from 9 to 10.

The hybridization results may be visualized using well known immunohistochemical staining methods to detect the labeling on the probe. When fluorescent labeled probes are used, the hybrids may be detected using an antibody against the fluorescent label which antibody may be conjugated with an enzyme. The fluorescent label may alternatively be detected directly using a fluorescence microscope, or the results may be automatically analyzed on a fluorescence-based image analysis system.

When biotin labeled probes are used, the hybrids may be detected using an antibody against the biotin label which antibody may be conjugated with an enzyme. If necessary, an enhancement of the signal can be generated using commercially available amplification systems such as the catalyzed signal amplification system for biotinylated probes.

Kits provided by the invention include kits for the expression analysis of human papillomavirus, for assessing the efficacy of an HPV-based disease, for the diagnosis of an HPV-based disease. The kits are provided with one or more high affinity probes of the invention, for example, probes identified by SEQ ID NOS. 1-14, and derivatives thereof, and instructions for use. For example, the kits may provide instructions for use of the probes in an in-situ hybridization assay. The kits may be specific for the detection of cervical specimens, for example, ThinPreps or Pap stained specimens. Other specimen types useful have been identified herein and are otherwise known.

### EXAMPLES

### Example 1

Detection of HPV RNA of Infected Cells in Cervical Carcinoma.

Obtain a paraffin embedded tissue section from high grade cervical carcinoma infected with HPV-16. The tissue sample may be frozen immediately after collection. The sample is placed in 100 mM phosphate, 4% formaldehyde for 12 hours followed by embedding in paraffin. At some later point, 4 μm sections are cut from the paraffin sample and transferred onto silanized microscope slides. The slides are then dried by incubating for 30 minutes at 60° C.

The slides are next deparaffinized and probed as described in Example 3 with the exception that a fluorescein labeled PNA probe according to Seq ID#12 is used to detect HPV-16 mRNA. The hybridization buffer contains 30% formamide v/v, 1% Triton-X 100 v/v, 10% dextran sulfate w/v, 0.2% polyvinylpyrillidone w/v, 0.2% ficoll 400 w/v, 0.1% sodium pyrophosphate w/v, 5 mM EDTA, 10 mM NaCl, 50 mM Tris pH 7.6 plus 50 nM PNA probe. The 1× wash buffer contains 1% Triton-X 100, 20 mM NaCl, 5 mM Tris pH 10.

### Example 2

Detection of mRNA of HPV Infected Cells from a Transformed Cell Line.

Obtain a tissue culture flask in which Caski cells have been recently passaged, and are growing, but which are not confluent. Remove growth media, and wash cells twice with phosphate buffer saline. Remove cells from the tissue culture flask by incubating with PBS containing trysin at 0.25% for 5 minutes at 37° C. Centrifuge cells in IEC Clinical centrifuge model 428 (or equivalent) at setting 5 for 5 minutes, wash pellet twice w/PBS. Resuspend cells in 4% paraformaldehyde/PBS. Allow cell suspension to stand at RT for 0.5-2 hours. Repellet and wash pellet twice w/PBS. Repellet and resuspend cells in 50% ethanol. Store cells at −20° C. until use. Resuspend cells by vortexing, prepare slides by spotting 50 uL cell suspension onto slide, then drying at 55° C. for 10 minutes.

The slides are next probed as described in Example 3 with the exception that a fluorescein labeled PNA probe according to Seq ID#12 is used to detect HPV-16 mRNA. The hybridization buffer contains 30% formamide v/v, 1% Triton-X 100 v/v, 10% dextran sulfate w/v, 0.2% polyvinylpyrillidone w/v, 0.2% ficoll 400 w/v, 0.1% sodium pyrophosphate w/v, 5 mM EDTA, 10 mM NaCl, 50 mM Tris pH 7.6 plus 50 nM PNA probe. The 1× wash buffer contains 1% Triton-X 100, 20 mM NaCl, 5 mM Tris pH 10.

### Example 3

Direct Fluorescence In Situ Detection of Glyceraldehyde Phosphate Dehydrogenase (GAPDH) RNA Targets.

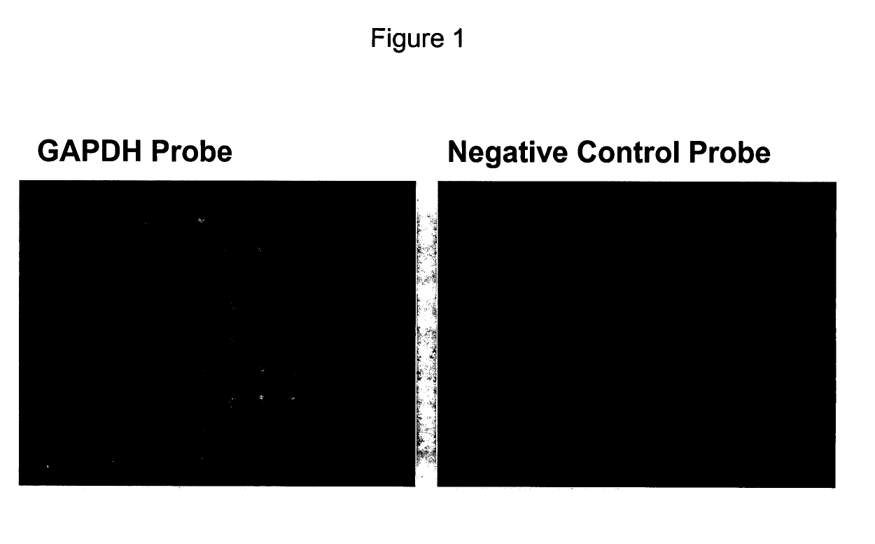

Paraffin embedded SiHa cells were obtained as prepared slides (DAKO Cytomation #0629). Slides were deparaffinized by 2×5 minute baths in xylene followed by 2×3 minute baths in 100% ethanol, followed by 2×3 minute baths in 96% ethanol, followed by 3×3 minute baths in deionized water (RNAse free). Detection of GAPDH mRNA followed through application of reagents from a commercially available detection kit (DAKO Cytomation #5201), except that signal amplification part involving enzyme-labeled antibody was not performed. Briefly, slides were treated with proteinase K diluted in TBS at room temperature for 30 minutes, then rinsed twice in water for 3 minutes each, followed by a 10 minute wash in reagent alcohol. After 5 minutes of air drying, a probe solution was applied to the slides which contained either a GAPDH mRNA specific PNA probe, or a control solution of random PNA probes of equal size. The probes were labeled with fluorescein, a green fluorescent fluorophore. Coverslips were placed over the respective probe solutions, and slides were incubated in a hybridization chamber at 55° C. for 90 minutes. Following hybridization, slides were soaked in 200 mL of 1× stringent wash solution (provided with kit) at 55° C. for 30 minutes. Slides were rinsed once with 1× TBS wash at RT for 1 minute. Slides were allowed to air dry, and then a drop of AdvanDx mounting media (KT001, AdvanDx, Woburn, Mass.) and a cover slip were applied. Visualization of the cells was performed on a fluorescent microscope equipped with a FITC/Texas Red dual band-pass filter. With reference to FIG. 1, the figure displays a 0.5 second exposure of slides observed on an Olympus BX-51 fluorescent microscope at 600× magnification. Images were obtained using a digital camera mounted on the microscope. As can be seen in FIG. 1 slides detected with the GAPDH probe produced bright (green) fluorescence which appeared to be concentrated more in the cytoplasm, than in the nucleus. The control probe gave a very weak nonspecific signal which was distributed evenly over the cells.

The results surprisingly showed that direct fluorescence in situ hybridization without the use of signal amplifications as otherwise instructed by the information provided with the reagents (package insert, K5201, DakoCytomation) and prior art resulted in unequivocal detection of GAPDH mRNA. GAPDH is a house-keeping gene with an medium expression level, indicating that the method is not restricted to certain highly expressed genes but generally applicable to genes with different expression levels. In this example paraffin embedded samples were used, but samples prepared using other sample preparation methods should be equally suitable as long as they preserve the mRNA.

What is claimed is:

1. A high affinity probe for expression analysis of an mRNA, comprising a nucleobase sequence complementary to a target sequence of an mRNA.

2. A high affinity probe for expression analysis of a cancer marker, comprising a nucleobase sequence complementary to a target sequence of the cancer marker.

3. The high affinity probe of claim 2, wherein the cancer marker is one or more of Brn-3a, Bcl-2, Her2-neu, p53, E6/E7, E1/E2 or a growth factor receptor.

4. A high affinity probe for expression analysis of human papillomavirus, comprising a nucleobase sequence complementary to a target sequence of human papillomavirus mRNA.

5. The high affinity probe of claim 1, wherein the high affinity probe is directly detectable.

6. The high affinity probe of claim 1, wherein the probe sequence is between about 8 to about 20 subunits in length.

7. The high affinity probe of claim 1, wherein the probe is labeled with at least one detectable moiety.

8. The high affinity probe of claim 1, wherein the detectable moiety is one ore more of a conjugate, a branched detection system, a chromophore, a fluorophore, a spin label, a radioisotope, an enzyme, a hapten, an acridinium ester, or a luminescent compound.

9. The high affinity probe of claim 1, wherein the probe is self-reporting.

10. The high affinity probe of claim 1, wherein the probe is a PNA Linear Beacon.

11. The high affinity probe of claim 1, wherein the probe further comprises a spacer or a linker.

12. The high affinity probe of claim 1, wherein in situ hybridization is used for analysis of the target sequence.

13. The high affinity probe of claim 4, wherein the target sequence is one or more of human papillomavirus E6 or E7 mRNA.

14. The high affinity probe of claim 4, wherein the target sequence is one or more of spliced human papillomavirus E6 or E7 mRNA.

15. The high affinity probe of claim 4, wherein human papillomavirus mRNA is human papillomavirus type 16.

16. The high affinity probe of claim 4, wherein human papillomavirus mRNA is human papillomavirus type 18.

17. The high affinity probe of claim 4, wherein human papillomavirus mRNA is human papillomavirus type 31.

18. The high affinity probe of claim 4, wherein human papillomavirus mRNA is human papillomavirus type 33.

19. The high affinity probe of claim 4, wherein human papillomavirus mRNA is human papillomavirus type 45.

20. The high affinity probe of claim 4, wherein the high affinity probe is a peptide nucleic acid (PNA) probe.

21. The high affinity probe of claim 4, wherein the high affinity probe is a locked nucleic acid (LNA) probe.

22. The high affinity probe of claim 4, wherein the probe comprises one or more of the following sequences: (HPV16 E6*): ATA TAG CTC ACG TCG (SEQ ID NO: 1); (HPV18 E6*): AGG CAC CTC TGT MG (SEQ ID NO: 2); (HPV31 E6*): ATA CAC CTC TGT TTC (SEQ ID NO: 3); (HPV33 E6*): ATA CAC CTC AGA TCG (SEQ ID NO: 4); (HPV35 E6*): ATA CAC CTC ACT CCG (SEQ ID NO: 5); (HPV16 E7*): TCC AAA GTA CGA ATG T (SEQ ID NO: 8); (HPV18 E7*): GTC TTC CM AGT ACG A (SEQ ID NO: 6; (HPV31 E7*): CTT GCA ATA TGC GAA TAT (SEQ ID NO: 10); (HPV33 E7*): GTA TGG TTC GTA GGT (SEQ ID NO: 9); (HPV35 E7*): TTC CM TTT ACG TAT GTC (SEQ ID NO: 7; (HPV16 E6 B) TCG CAG TM CTG TTG C (SEQ ID NO: 11); (HPV16 E6 C) TCA CGT CGC AGT MC (SEQ ID NO: 12); (16E6E7 A) TAA TAC ACC TCA CGT (SEQ ID NO: 13); or (16E6E7 B) GTT AAT ACA CCT CAC (SEQ ID NO: 14).

23. A high affinity probe set, comprising two or more high affinity probes identified in claim 1, wherein the probes are used for the analysis of mRNA expression.

24. The high affinity probe set of claim 23, wherein the probes are used for the analysis of human papillomavirus in a sample.

25. The high affinity probe set of claim 23, wherein the high affinity probes are differently labeled for independent analysis.

26. The high affinity probe set of claim 23, wherein the high affinity probes are directly detectable.

27. A method for expression analysis of mRNA, comprising contacting a cytological specimen with a high affinity probe of claim 1 under conditions suitable for in situ hybridization.

28. A method for expression analysis of a cancer marker, comprising contacting a cytological specimen with a high affinity probe of claim 1 under conditions suitable for in situ hybridization.

29. The method of claim 28, wherein the cancer marker is one or more of Brn-3a, Bcl-2, Her2-neu, p53, E6/E7, E1/E2 or a growth factor receptor.

30. A method for expression analysis of human papillomavirus in a sample comprising:
(a) contacting a cytological specimen with a high affinity probe of claim 1,
(b) incubating the sample with the high affinity probe; and
(c) detecting fluorescence from the sample, wherein the level of fluorescence is indicative of the expression of the human papillomavirus within the sample.

31. The method of claim 30, wherein the in situ hybridization is fluorescence in situ hybridization.

32. The method of claim 27, wherein the probes are directly detectable.

33. The method of claim 27, wherein two or more high affinity probes are used.

34. The method of claim 27, wherein the probes are similarly labeled.

35. The method of claim 27, wherein the probes are differently labeled.

36. The method of claim 27, wherein the detectable moiety is a hapten, wherein the hapten is detected by an enzyme-antibody conjugate capable of producing signal amplification.

37. The method of claim 35, wherein the enzyme in the enzyme-conjugate is selected from a group consisting of alkaline phosphatase, horseradish peroxidase, and soybean peroxidase.

38. The method of claim 35, where the signal amplification is by tyramide signal amplification.

39. The method of claim 30, wherein the probes are a mixture of type-specific HPV probes.

40. The method of claim 30, wherein the stage of HPV-based disease is assessed.

41. The method of claim 30, further comprising a second assay of HPV-based disease.

42. The method of claim 40, wherein the second assay is histological staining.

43. The method of claim 42, wherein the histological staining is Pap staining.

44. The method of claim 40, wherein the second assay is performed on the same cytological specimen.

45. The method of claim 40, wherein the second assay is performed simultaneously with the contacting a cytological specimen with a high affinity probe.

46. The method of claim 40, wherein the second assay is performed prior the contacting a cytological specimen with a high affinity probe.

47. A method of assessing the risk of developing HPV-based disease or cancer, comprising:
a) contacting a sample from a subject with one or more high affinity probes complementary to a target sequence of human papillomavirus mRNA, and
b) determining the presence of bound probe.

48. The method of claim 45, wherein the one or more probes are SEQ ID NOS: 1-14 or derivatives thereof.

49. The method of claim 45, wherein the one or more probes are differentially labeled.

50. The method of claim 45, wherein the probes are similarly labeled.

51. The method of claim 45, wherein the determining the presence of bound probe is by in situ hybridization.

52. The method of claim 45, further comprising removing any unbound probe prior to determining the presence of bound probe.

53. The method of claim 45, further comprising correlating the presence or absence of papillomavirus mRNA with a risk of developing HPV-based disease.

54. The method of claim 53, wherein the correlating distinguished between level of risk.

55. The method of claim 54, wherein levels of risk comprise no risk, moderate risk, and high risk.

56. The method of claim 55, wherein no risk correlates with no detectable HPV.

57. The method of claim 55, wherein moderate risk correlates with detection an HPV infection but no detectable HPV-based disease.

58. The method of claim 55, wherein high risk correlates with the detection of high levels of HPV infection.

59. A method for selecting subjects for treatment for HPV-based disease, comprising
a) contacting a sample from a subject with one or more high affinity probes complementary to a target sequence of human papillomavirus mRNA,
b) determining the presence of bound probe, and
c) correlating the presence of papillomavirus mRNA with a need for treatment for HPV-based diseases.

60. The method of claim 59, further comprising obtaining a sample from the subject.

61. The method of claim 59, further comprising treating a subject for HPV-based diseases based on the presence of papillomavirus mRNA.

62. A method for monitoring the efficacy of an HPV-treatment, comprising
a) determining a pre-treatment level of HPV infection,
b) administering an HPV infection treatment, and
c) determining a post-treatment level of HPV infection after an initial period of treatment.

63. The method of claim 62, further comprising correlating a decrease in the level of infection with an efficacy of treatment.

64. The method of claim 62, wherein the pre-treatment and post-treatment levels of HPV infection are determined by contacting a sample from a subject with one or more high affinity probes complementary to a target sequence of human papillomavirus mRNA and detecting the presence of bound probe.

65. A method for the expression analysis of human papillomavirus by in situ hybridization, comprising:
a) contacting the sample with at least one high-affinity probe that is substantially complementary to a portion of a HPV mRNA.
b) incubating the sample with the high affinity probe; and
c) detecting the fluorescence of the sample, wherein the level of fluorescence is indicative of the presence and/or amount of mRNA within individual cell of the sample.

66. The method of claim 65, wherein the presence of the HPV is by the detection of the expressed mRNA.

67. The method of claim 65, further comprising correlating a level of fluorescence with a down-regulation of expression of HPV mRNA.

68. The method of claim 65, further comprising correlating a level of fluorescence with an up-regulation of expression of HPV mRNA.

69. The method of claim 65, further comprising a comparison of expression of two or more mRNAs.

70. A method of diagnosing or predicting an HPV-based disease in a subject, comprising:
a) determining a level of HPV infection by contacting a sample from a subject with one or more high affinity probes complementary to a target sequence of human papillomavirus mRNA, and determining the presence of bound probe;
b) comparing the level, to a standard level; and
c) correlating a modulated level in the cell from the subject with an indication of an HPV-based disease.

71. The method of claim 70, wherein the standard level is the corresponding level in a reference cell or population of cells.

72. The method of claim 71, wherein the reference cell is one or more of the following, cells from the subject, cultured cells, cultured cells from the subject, cells from the subject pre-treatment, cells from a second subject not suspected or showing no signs of an HPV-based disease.

73. The method of claim 70, further comprising obtaining a cell sample from the subject.

74. The method of claim 70, further comprising reporting the level or correlations thereof to the subject or a health care professional.

75. A kit for expression analysis of human papillomavirus comprising one or more high affinity probes and instructions for use in an in-situ hybridization assay.

76. The kit of claim 75, wherein the kit is used to examine cervical specimens.

77. The kit of claim 76, wherein the cervical specimens are ThinPreps.

78. The kit of claim 76, wherein the cervical specimens have been Pap stained

79. A kit for the assessing the stage of HPV-based diseases, comprising one or more high affinity probes for expression analysis of human papillomavirus, wherein the proved comprises a nucleobase sequence complementary to a target sequence of human papillomavirus mRNA.

80. The kit of claim 79, wherein the high affinity probes are selected from one or more of the probes identified by SEQ ID NOS: 1-14.
